(19) United States
(12) Patent Application Publication
(10) Pub. No.: US 2022/0273579 A1
(43) Pub. Date: Sep. 1, 2022

(54) ADHESIVE DRUG DELIVERY FILM AND A MICROPARTICLE COMPRISING THEREOF

(71) Applicant: AMD PHARMA LTD., Tel Aviv (IL)

(72) Inventor: Adel PENHASI, Modiin (IL)

(21) Appl. No.: 17/635,388

(22) PCT Filed: Aug. 16, 2020
(86) PCT No.: PCT/IL2020/050902

§ 371 Date: Feb. 15, 2022

Related U.S. Application Data
(60) Provisional application No. 62/887,816, filed on Aug. 16, 2019.

(51) Int. Cl. A61K 9/50; A61K 9/00

(52) U.S. Cl. CPC A61K 9/5031; A61K 9/5026; A61K 9/0053

(57) ABSTRACT

According to some embodiments, there is provided herein a microparticle for the administration of a pharmaceutical composition at the upper gastrointestinal tract comprising: a core comprising at least one pharmaceutical composition; at least one excipient a first coating layer comprising a bioadhesive material comprising a combination of PEO and PVP.

### FIELD OF THE INVENTION

The present invention relates to the field of pharmaceutics, specifically to an adhesive drug delivery film and to a microparticle comprising thereof specifically aimed at the targeted delivery of pharmaceutical compositions at the gastrointestinal tract.

### BACKGROUND OF THE INVENTION

Controlled release systems for drug delivery are often designed to administer drugs in specific areas of the gastrointestinal (GI) tract. Often the challenge with certain drugs is the specific area of absorption, wherein beyond this area, the drug may have little or no absorption into the human body.

The desired absorption rate and extent results in reaching a target blood level of the active substance of the drug, having direct correlation to the safety and effectiveness of the drug.

Some drugs possess a narrow window of absorption in the GI tract, making it extremely difficult to provide effective controlled release compositions containing these drugs, and often resulting in final pharmaceutical compositions that need to be administered several times a day or have a high risk of toxicity or substantial side effects.

Also, beyond the issue of the specific location of the absorption of a drug, there is also importance to the duration of time that the drug is situated in the desired location. With some drugs, especially neuroactive drugs, the patient may suffer from side effects or lower efficacy if blood serum concentrations vary considerably.

Within the entire GI tract, there are specific regions which might be more complicated for drug absorption in comparison to other regions, for example, the stomach which is characterized by an acidic environment and a constant secretion of mucosa on the stomach walls, and for example, the duodenum which is considered the optimal absorption site for several drugs, e.g., due to the secretion of bile, is actually a rather short section within the entire GI tract.

Thus, there is a need for specific compositions and methods for controlling the absorption of pharmaceutical agents transferred through specific regions within the GI tract.

### SUMMARY OF THE INVENTION

According to some demonstrative embodiments, there is provided herein a bioadhesive microparticle comprising one or more pharmaceutical compositions, wherein the microparticle comprises one or more pharmaceutical agents which are to be absorbed into the human body via one or more portions of the GI tract, preferably, via the upper GI tract.

According to some demonstrative embodiments, there is provided herein a microparticle for the administration of a pharmaceutical composition at the upper gastrointestinal tract comprising:
a core comprising
   at least one pharmaceutical composition;
   at least one excipient
a first coating layer comprising
   a bioadhesive material comprising a combination of PEO and PVP.

According to some demonstrative embodiments, the core may further comprise a hydrophilic component and a binder.

According to some demonstrative embodiments, the first layer may further comprise channel forming agent.

According to some demonstrative embodiments, the bioadhesive material may comprise high molecular weight PVP above 30 KD (Kilo Dalton) and high molecular weight Polyethylene oxide (PEO) above 20 KD.

According to some demonstrative embodiments, the ratio between PEO and PVP is 60:40, respectively.

According to some demonstrative embodiments, the channel forming agent may comprise a polymer selected from the group including low molecular weight Polyethylene glycol (PEG), low molecular weight PolyEthylene oxide (PEO) below 20 KD, low molecular weight Polyvinylpyrrolidone (PVP) below 30 KD, low molecular weight Polyvinyl alcohol (PVA) below 31 KD, low viscosity grade Sodium Carboxymethyl cellulose (Na-CMC) (7L and 7L2), low viscosity Hydroxyethyl cellulose (HEC) (250 JR or 250 LR).

According to some demonstrative embodiments, the product may comprise microparticles of different diameters or of substantially similar diameter.

According to some demonstrative embodiments, there is provided herein a process for preparing the core of the microparticle of the present invention by granulation or by extrusion.

### DESCRIPTION OF THE DRAWINGS

FIG. 1 depicts a Tensile bioadhesion testing diagram, according to some demonstrative embodiments of the present invention.

### DETAILED DESCRIPTION OF THE INVENTION

According to some demonstrative embodiments, there is provided herein a bioadhesive microparticle comprising one or more pharmaceutical compositions, wherein the microparticle comprises one or more pharmaceutical agents which are to be absorbed into the human body via one or more portions of the GI tract, preferably, via the upper GI tract.

Drugs are usually classified using the Biopharmaceutical Classification System (BCS), which categorizes pharmaceutical compositions for oral administration into four main classes depending on their solubility and their permeability through the intestinal wall.

According to the BCS, the classifications are as follows:

### Class I—High Permeability, High Solubility

### Class II—High Permeability, Low Solubility

### Class III—Low Permeability, High Solubility

### Class IV—Low Permeability, Low Solubility.

According to some embodiments, the term “pharmaceutical composition”, also referred to herein as “the active substance” or “active pharmaceutical ingredient (API)” may include to any suitable drug from Classes I, II, II and IV, Preferably, from Class I and/or II.

According to some embodiments, the pharmaceutical composition may be selected from the group including caffeine, carbamazepine, fluvastatin, Ketoprofen, Metoprolol, Naproxen, Propranolol, Theophylline, Verapamil, Diltiazem, Gabapentin, Levodopa, Divalproex sodium, itraconazole and its relatives, fluoconazole, terconazole, ketoconazole, and saperconazole, griseofulvin and related compounds such as griseoverdin, anti malaria drugs, immune system modulators e.g. cyclosporine, cardiovascular drugs (e.g. digoxin and spironolactone), ibuprofen, danazol, albendazole, clofazimine, acyclovir, carbamazepine, proteins, peptides, polysaccharides, nucleic acids, nucleic acid oligomers, viruses, Neomycin B, Captopril, Atenolol, Valproic Acid, Stavudine, Salbutamol, Acyclovir, Methotrexate, Lamivudine, Ergometrine, Ciprofloxacin, Amiloride, Caspofungin, Clorothiazide, Tobramycin, Cyclosporin, Allopurinol, Acetazolamide, Doxycyclin, Dapsone, Nalidixic Acid, Sulfamethoxazole, Tacrolimus, And Paclitaxel.

According to some embodiments, the bioadhesive microparticle of the present invention may include one or more pharmaceutical agents together with at least one bioadhesive binder and optionally at least one hydrophilic component.

According to some demonstrative embodiments, the term “microparticle” may include any suitable small sized particles including for example, granules, pellets, particulates, grains, spheres and the like.

According to some embodiments, the use of microparticles according to the present embodiments allows for a larger surface area in comparison to a tablet or a large particle, which, for example, directly affects the rate and/or extent of absorption of the API.

According to some embodiments, the term “bioadhesive”, “bioadhesive polymer” or “bioadhesive material” may refer to the bioadhesive compositions disclosed herein, including materials that contain one or more additional components in addition to the bioadhesive polymers and bioadhesive compositions of the invention.

According to some embodiments, bioadhesives may also include blends of one or more bioadhesive polymers.

In some embodiments, the term “bioadhesive polymers” may be used to refer to both compositions where the polymer itself is bioadhesive, as well as compositions where a non- or poorly bioadhesive polymer is combined with a compound that imparts bioadhesive properties to the composition as a whole, as described in detail herein.

A bioadhesive material may generally refer to a material possessing the ability to adhere to a biological surface for an extended period of time. Bioadhesion requires a contact between the bioadhesive material and the receptor surface, such that the bioadhesive material penetrates into the crevice of the surface (e.g. tissue and/or mucus).

According to some demonstrative embodiments, the bioadhesive may include any high molecular weight crosslinked polyacrylic acid polymers. According to some embodiments, such polymers may differ by crosslink density and can be grouped into the following categories.

I. Polymers of acrylic acid crosslinked with allyl sucrose or allyl pentaerythritol (also known as Carbopol homopolymers).

II. Polymers of acrylic acid and C10-C30 alkyl acrylate crosslinked with allyl pentaerythritol (also known as Carbopol copolymers).

III. Carbomer homopolymer or copolymer that contains a block copolymer of polyethylene glycol and a long chain alkyl acid ester (also known as Carbopol interpolymers).

According to some demonstrative embodiments, the bioadhesive may be comprised of a combination of at least two neutral polymers, having a synergistic effect when combined, including, for example, PVP and PEO.

According to some embodiments, preferably the bioadhesive material comprises high molecular weight PVP above 30 KD and high molecular weight Polyethylene oxide (PEO) above 20 Kilo Dalton.

According to some embodiments, using a high molecular weight polymers, for example, high molecular weight PEO, is preferable since it does not dissolve immediately while the polymer is still able to absorb a high volume of water which in turn enhances the wettability of the microparticle according to the present invention.

According to some demonstrative embodiments, the microparticle may further include a channel forming agent, to cause the formation of pores in the microparticle, e.g., to cause the initiation of the extrusion of the pharmaceutical composition from the microparticle into the surrounding environment.

According to some embodiments, the term “channel forming agent” may include any suitable water soluble polymer including, for example Low molecular weight Polyethylene glycol (PEG), PolyEthylene oxide (PEO), preferably a low molecular weight of PEO, Polyvinylpyrrolidone (PVP), Polyvinyl alcohol (PVA), Sodium Carboxymethyl cellulose (Na-CMC), Hydroxyethyl cellulose (HEC).

According to some embodiments, the channel forming agent may preferably be a low molecular weight PVP of up to 30 Kilo Dalton.

The duodenum may be a location to which materials can hardly adhere to, since the villi of the duodenum have a leafy-looking appearance, which is a histologically identifiable structure and the Brunner's glands, which secrete mucus, are found in the duodenum only. The duodenum wall is also composed of a very thin layer of cells that form the muscularis mucosae.

Other areas in the GI tract which are difficult for the adherence of materials may include the stomach, which has an acidic and mucosal environment, and also the jejunum.

According to some demonstrative embodiments, the unique combination of the components of the microparticle of the present invention may be especially beneficial in adherence to the duodenal wall lining, as well as to the Stomach and/or jejunum lining.

According to some embodiments, the microparticle may be an uncoated particle, including the one or more pharmaceutical agents together with at least one bioadhesive binder, at least one hydrophilic component and optionally a binder.

According to some embodiments, the microparticle may be a coated particle, including a core comprising the one or more pharmaceutical agents; optionally a hydrophilic component and optionally a binder;

A first layer comprising bioadhesive polymers.

According to some demonstrative embodiments, the combination of some important parameters such as the large surface area of the microparticle, which is derived from the relatively small particle size of microparticles and the bio-adhesive polymer(s), is specifically beneficial in an immediate and as fast wetting process of the drug delivery system after exposure to the gastric fluid and thus the adherence to the upper parts of the GI tract such as stomach, duodenal and the jejunum region.

According to some demonstrative embodiments, there is provided herein a composition, also referred to herein as a product, comprising a plurality of microparticles according to the present invention.

According to some demonstrative embodiments, the microparticles contained within the composition may have a substantially similar size and/or diameter, for example, to allow for a specific unified release rate.

According to some embodiments, the size of the microparticles determines the rate and/or extent of the absorption of the active substance.

Brunauer-Emmett-Teller (BET) theory aims to explain the physical adsorption of gas molecules on a solid surface and serves as the basis for an important analysis technique for the measurement of the specific surface area of materials.

According to some embodiments, the microparticle size may be in the range between 100 to 1500 microns, preferably between 300 to 1200 microns and most preferably between 500 to 1000 microns.

According to some embodiments, upon exposure to an aqueous environment in the human body, a plurality of pores may be created on the surface of the microparticle of the present invention, for example, due to the dissolution of the channel forming agent(s).

The size, shape, pore volume, pore distribution of the microparticle, may directly affect the surface area and consequentially, the rate and/or extent of release of the pharmaceutical composition from the microparticle.

According to some embodiments, there is provided herein a delivery system containing a variety of microparticles having different sizes, optionally comprising soluble or insoluble polymers, allowing for an immediate release or sustained release or a combination thereof.

According to some embodiments, the combination of PEO and PVP possesses unique and beneficial adherence effect.

According to some embodiments, the ratio between PEO and PVP may between 0:100 to 100:0, preferably between 70:30 to 0:100, more preferably between 70:30 to 50:50, most preferably 60:40, respectively.

### Example 1

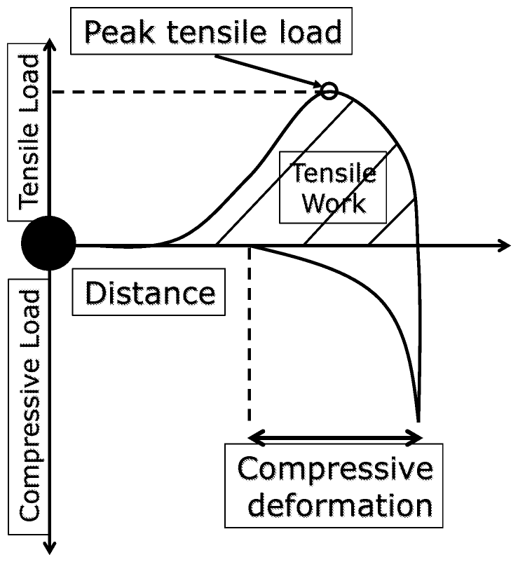

Reference is made to FIG. 1, which exhibits Tensile bioadhesion testing diagram.

An in-vitro bio-adhesion test was conducted on various films which contained different ratios of polyethylene oxide (PEO) to Polyvinylpyrrolidon (PVP), wherein the adhesion test was conducted as per the diagram of FIG. 1.

The results of the tests are as follows:

| | | | Results | |
|---|---|---|---|---|
| | | | [Adhesion Test] | |
| Sample No. | PEO | PVP | Force | Work Avg |
| 1.1 | 100 | — | 180.396 | 83.621 |
| 1.2 | 90 | 10 | 50.828 | 83.562 |
| 1.3 | 80 | 20 | 58.241 | 95.350 |
| 1.4 | 70 | 30 | 322.804 | 212.074 |
| 1.5 | 60 | 40 | 359.2 | 222.720 |
| 1.6 | 50 | 50 | 349 | 252.958 |
| 1.7 | — | 100 | 253.807 | 136.408 |

While this invention has been described in terms of some specific examples, many modifications and variations are possible. It is therefore understood that within the scope of the appended claims, the invention may be realized otherwise than as specifically described.

1. A microparticle for the administration of a pharmaceutical composition at the upper gastrointestinal tract comprising:
a core comprising
at least one pharmaceutical composition;
at least one excipient
a first coating layer comprising
a bioadhesive material comprising a combination of PEO and PVP.

2. The microparticle of claim 1, wherein said core further comprises a hydrophilic component and a binder.

3. The microparticle of claim 1, wherein said first layer further comprises channel forming agent.

4. The microparticle of claim 1, wherein said bioadhesive material comprises high molecular weight PVP above 30 KD high molecular weight Polyethylene oxide (PEO) above 20 Kilo Dalton.

5. The microparticle of claim 4, wherein the ration between PEO and PVP is 60:40, respectively.

6. The microparticle of claim 3, wherein said channel forming agent comprises a polymer selected from the group including low molecular weight Polyethylene glycol (PEG), low molecular weight PolyEthylene oxide (PEO) below 20 KD, low molecular weight Polyvinylpyrrolidone (PVP) below 30 KD, low molecular weight Polyvinyl alcohol (PVA) below 31 KD, low viscosity grade Sodium Carboxymethyl cellulose (Na-CMC) (7L and 7L2), low viscosity Hydroxyethyl cellulose (HEC) (250 JR or 250 LR).

7. A product comprising the microparticle of claim 1, wherein said product comprises microparticles of different diameters.

8. A product comprising the microparticle of claim 1, wherein said product comprises microparticles of substantially similar diameter.

9. A process for preparing the core of the microparticle of claim 1 comprising granulation or extrusion.
